(19) United States
(12) Patent Application Publication
(10) Pub. No.: US 2026/0256640 A1
(43) Pub. Date: Sep. 3, 2026

(54) SYSTEMS AND METHODS TO TREAT RETINAL DEGENERATION DISORDERS

(71) Applicant: DANDELION SCIENCE CORP., New York, NY (US)

(72) Inventors: Adam HANINA, New York, NY (US); Ajay VERMA, Wayland, MA (US); Gopal K. CHOPRA, New York, NY (US); Dan NEMRODOV, Jersey City, NJ (US); Ekaterina MALAKHOVA, Highlands Mills, NY (US)

(21) Appl. No.: 19/165,433

(22) PCT Filed: Mar. 13, 2024
(86) PCT No.: PCT/US2024/019749

§ 371 Date: Sep. 15, 2025

Related U.S. Application Data
(60) Provisional application No. 63/490,235, filed on Mar. 14, 2023.

(51) Int. Cl. A61H 5/00; G16H 20/40

(52) U.S. Cl. CPC A61H 5/00; G16H 20/40; A61H 2201/10; A61H 2201/5007; A61H 2201/5043

(57) ABSTRACT

Systems and methods for treating eye disorders, such as retinal degeneration disorders, are disclosed. Adapted visual neuromodulatory codes are retrieved. The adapted visual neuromodulatory codes are adapted to produce neuromodulatory responses in the visual cortex. The adapted visual neuromodulatory codes are output to an electronic display of a device viewable by a patient.

### CROSS-REFERENCE TO RELATED APPLICATION(S)

The present application claims priority to U.S. Provisional Patent Application No. 63/490,235, filed Mar. 14, 2023, which is hereby incorporated by reference in its entirety.

### BACKGROUND

### Technical Field

The present disclosure generally relates to systems and methods to treat retinal degeneration disorders and, in particular, disorders involving progressive retinal degeneration.

### Description of the Related Art

Retinal degeneration disorders are a group of diseases that affect the retina, the thin layer of tissue at the back of the eye that is responsible for capturing and transmitting visual information to the brain. Currently, there are limited treatment options available for retinal degeneration disorders, and none of these options can cure or reverse the damage caused by the disease. Treatments may include medications and, in some cases, surgery. However, these treatments are often only partially effective and may have significant side effects. Retinal degeneration disorders can significantly impact an individual's quality of life and independence.

Age-related macular degeneration (AMD) is an example of a visual disorder which causes loss of vision in a manner which affects the eye but does not substantially impair the visual processing centers in the brain, e.g., the visual cortex. AMD is a leading cause of blindness in people over 50 years of age. The condition initially affects the macula, which is the part of the retina responsible for central vision. AMD can cause irreversible damage to the macula, leading to a progressively increasing loss of central vision. However, the peripheral vision remains intact early on in most cases. AMD is a common cause of blindness and visual impairment, and it has a significant impact on public health. For example, in terms of prevalence, AMD is a common condition, particularly among older adults. According to the National Eye Institute, AMD is the leading cause of blindness among Americans aged 65 and older, with an estimated 11 million people in the United States affected by the disease. AMD can cause significant visual impairment and disability, affecting a person's ability to perform daily activities such as reading, driving, and recognizing faces.

The economic burden of AMD is substantial, including the costs of medical care, rehabilitation, and lost productivity. According to some estimates, the annual direct medical costs of AMD in the United States are about $6.7 billion. AMD is associated with increased healthcare utilization, including hospitalizations, emergency room visits, and outpatient visits. This can place a strain on the healthcare system and increase healthcare costs. Thus, age-related macular degeneration has a significant impact on public health—affecting millions of people in the United States and around the world. The disease can cause significant disability, economic burden, and healthcare utilization, highlighting the need for effective prevention and treatment strategies.

About 90 percent of all people with Age-related Macular Degeneration have “dry” AMD, a condition in which layers of the macula (including the photoreceptors and the retinal pigment epithelium) get progressively thinner and atrophy, functioning less and less as they do. There are currently no approved drugs for the dry form of AMD.

About ten percent of all cases of Age-related Macular Degeneration become “wet” AMD. Typically, a person has dry AMD first and progresses toward the wet form. In the wet form of AMD new blood vessels grow in the choroid layer behind the retina. This condition is called choroidal neovascularization. The new vessels are weak, and they leak fluid, lipids and blood. The leaking gets into the layers of the retina, including the layers of the macula, and can cause scar tissue to form and retinal cells to stop functioning. Although some drugs that block neovascularization can be effective in slowing the progression of the wet form of AMD, there are some problems associated with their use as a treatment for the disease. While drugs such as anti-vascular endothelial growth factor (VEGF) agents have been shown to be effective in reducing the growth of abnormal blood vessels and leakage of fluid in the macula, they do not work for all patients. Some patients may not respond well to the drugs, or may experience a relapse of the disease after initial treatment.

Diabetic retinopathy is another example of a visual disorder which causes loss of vision in a manner which affects the eye but does not substantially impair the visual processing centers of the brain. Diabetic retinopathy is a complication of diabetes that affects the blood vessels in the retina, leading to damage and vision loss. Currently, there are no effective therapies for diabetic retinopathy, and the available treatments are aimed at managing the symptoms and slowing the progression of the disease. One of the challenges in treating diabetic retinopathy is that the disease is multifactorial and involves multiple mechanisms that contribute to its development and progression, such as oxidative stress, inflammation, and abnormal growth of blood vessels. Conventional therapies do not adequately address these mechanisms.

Retinitis pigmentosa (RP) is a group of genetic disorders that cause the gradual deterioration of the retina's photoreceptor cells, which are responsible for sensing light and transmitting visual information to the brain. This can result in progressive loss of peripheral vision, night blindness, and eventually, central vision loss.

Computer-based visual therapy is a treatment option for individuals with retinal degeneration, including AMD and retinitis pigmentosa. The goal of computer-based visual therapy is to improve visual function and quality of life by stimulating the remaining healthy cells in the retina and retraining the brain to better interpret visual information.

Vision stimulation is a rehabilitation treatment approach that involves the use of specific visual stimuli, e.g., letters, numbers, or magnification to improve visual function in patients with AMD. The goal of vision stimulation therapy is to increase the activity of the remaining healthy retinal cells, which can help compensate for the loss of function caused by AMD. Another approach, pulsed light therapy, involves the use of specific light frequencies and intensities to stimulate the retina. The therapy is typically delivered using a specialized device that emits high-intensity light pulses at specific frequencies. Another approach, electrotherapy, involves the use of electrical stimulation to promote neural plasticity in the visual cortex. The therapy is typically delivered using a specialized device that delivers low-intensity electrical currents to the retina. While vision stimulation therapy cannot restore lost vision, it can help patients adapt to the loss of vision caused by AMD.

Visual acuity tests are a common tool used to diagnose and monitor degenerative eye conditions such as age-related macular degeneration, diabetic retinopathy, and glaucoma. One common type of visual acuity test is the Snellen chart test, which involves standing a particular distance away from a chart or video monitor with letters of various sizes and trying to read the smallest letters possible. Another type of visual acuity test is the Early Treatment Diabetic Retinopathy Study (ETDRS) chart test, which is similar to the Snellen chart but is designed to be more precise and sensitive (the chart has rows of letters, with each row consisting of letters that are progressively smaller and harder to read). Another type of acuity test, specifically for macular degeneration, is the Amsler grid test, which involves looking at a grid of straight lines and trying to identify any areas where the lines appear distorted or missing, which can indicate damage to the macula. Patients with glaucoma may undergo a visual field test, which involves staring at a central point and identifying flashing lights in their peripheral vision.

### SUMMARY

Disclosed embodiments provide a digital therapeutic for the treatment of retinal degeneration disorders, such as age-related macular degeneration (AMD) or Diabetic Retinopathy. Disclosed systems and methods, in effect, exercise the visual pathway from eye to visual cortex.

AMD, for example, is a form of neurodegeneration that leads to the loss of central vision, resulting in blurry or wavy areas in the central field of vision. The diagnosis of AMD is usually done via a dilated eye exam. The proposed digital therapeutic is a neurofunctional therapeutic that aims to recruit existing pathways by presenting new types of visual information with a focus on enhancing the perception of remaining visual capabilities such as peripheral vision in AMD. It is a form of digital exercise that can be administered on any screen with or without neurofeedback. In particular aspects, the treatment may be directed to the functional retina which is not associated with a focal lesion at the retinal and cortical level, whereas, at the cortical level, the treatment may be directed to the whole functional retina.

In disclosed embodiments, empirically designed visual stimuli can be used to exercise visual pathways and cortical processing centers to enhance peripheral vision perception. This may include presenting visual stimuli that are specifically designed to activate and strengthen the neural pathways and processing centers that are responsible for specific portions of the field of vision, e.g., peripheral vision. The approaches disclosed herein may also be applicable to other areas of the cortex such as the frontal eye fields (FEF), ventral intraparietal (VIP) areas, fusiform gyrus and parahippocampal gyrus. Such cortical regions are responsible for visual attention, motion, and other contexts for visual perception. Therefore, these approaches may involve more than the peripheral vision, in which case the area to be considered may be peripheral to the focal lesion, but not necessarily limited to peripheral vision. In disclosed embodiments, the stimuli may be designed to take advantage of the binocular nature of vision with each eye's retina conveying info to each brain hemisphere. In such a case, the visual decline in one half of a visual field due to lesions in one eye's retina may be compensated by enhancing the functioning of the receiving cortical region by visual stimuli presented to the other eye.

In disclosed embodiments, the development of dynamic neuromodulatory stimuli, e.g., generative videos, is based at least in part on neural objectives relating to occipital processing, with the specific goal of adapting to peripheral function. Eye tracking may be used to determine peripheral re-training, and the video generates information that allows for improved peripheral function. The system helps to retrain the way the eye captures information, with saccades influencing this retraining. Generative artificial Intelligence (AI) can be used to map and optimize peripheral vision function, particularly visual function that is peripheral to a focal lesion, including color and movement in the peripheral vision. As AMD leads to a loss of cones, the system relies on rod vision, making it a form of neurofunctional enhancement to achieve visual recovery. In disclosed embodiments, the digital therapeutic can be personalized for each individual patient based on the type of AMD with which they are diagnosed.

In disclosed embodiments, the region of a focal lesion can be determined based at least in part on measured neural activity to recognize active and functional areas of the retina. Depending on where the active and functional areas lie in the retinal field, the dynamic visual stimulation can be adapted to achieve specific neural responses in the visual cortex.

In disclosed embodiments, the therapeutic approaches broadly treat various focal or diffuse lesions by recruiting existing functional circuits and enhancing the efficacy of those circuits to provide acute and chronic long-term improvement in functional vision, which may include expansion of visual field and improvement in acuity and recognition. For example, the deterioration of vision in Glaucoma is also progressive like AMD, but the pattern is opposite, with the periphery usually involved first and then the center. Diabetic neuropathy (i.e., retinopathy) has its own progressively deteriorating pattern. Disclosed embodiments are broadly aimed at enhancing the functioning of the “processor” or “receiver,” i.e., the brain and, in particular the thalamus and cerebral cortex, to compensate for a faulty “sensor,” i.e., the retina and optic nerve.

Disclosed embodiments can leverage the “Dandelion Chain” for generalizability as well as a “Content Lens” implementation overlaid on other screen content, as disclosed, for example, in Patent Cooperation Treaty (PCT) Application No. PCT/US2021/049080, filed Sep. 3, 2021, entitled “Artificial Intelligence—Guided Visual Neuromodulation for Therapeutic or Performance-Enhancing Effects; PCT Application No. PCT/US2022/77207, filed Sep. 28, 2022, entitled “Systems and Methods for Generating Spatiotemporal Sensory Codes”; and U.S. Provisional Patent Application No. 63/324,395, filed Mar. 28, 2022, entitled “Systems and Methods to Provide Dynamic Neuromodulatory Graphics,” all of which are incorporated herein by reference in their entirety

In disclosed embodiments, generative letter recognition and object recognition can be used in peripheral vision to train it to read and identify objects such as traffic lights, faces and places. This training helps to identify things that the peripheral vision was not used to doing, thereby improving general vision and visual acuity. Measures of success in visual acuity may be done using standard measurement techniques and may include particular milestones and/or thresholds, such as, for example, a two-line improvement on an eye chart.

In embodiments, “Dandelion Chain” algorithms are used to generate and optimize visual neuromodulatory codes to produce physiological responses having therapeutic or performance-enhancing effects. These algorithms may be implemented in two stages: an “inner loop” which optimizes visual neuromodulatory codes through biomedical sensor feedback to maximize the therapeutic impact for an individual subject or group of subjects; and an “outer loop” which uses various processing techniques to generalize the effectiveness of the visual neuromodulatory codes produced by the inner loop for the general population of users.

In embodiments, to maximize the chances of discovering responses that are consistent across subjects, optimization may be carried out on a group basis, in which case a group of subjects is presented simultaneously with visual images in the form of dynamic visual neuromodulatory codes, e.g., in video form. The bio-responses of the group of subjects are aggregated and analyzed in real time to determine which stimulation parameters (i.e., the parameters used to generate the visual neuromodulatory codes) are associated with the greatest response. The system optimizes the stimuli, readjusting and recombining the visual parameters to quickly drive the collective response of the group of subjects in the direction of greater response. Such group optimization increases the chances of evoking ranges of finely graded responses that have cross-subject consistency. This, in effect, creates new homunculus, i.e., a topographic representation of the eye along the postcentral gyrus of the parietal lobe, whereby retinal and functional response data are assembled that can be mapped to the cortex. In other words, rather than merely performing regional circuit linking to the cortex, the system is performing functional mapping to the cortex.

Embodiments may include techniques such as transfer and ensemble learning using artificial intelligence (AI), such as machine learning models and neural networks, e.g., convolutional neural networks, deep feedforward artificial neural networks, and adversarial neural networks, to develop better algorithms and produce generalizable therapeutic treatments. Instead of trying to create a perfect model of the brain, there is, in effect, a “chaining together” of a vast number of users to identify therapeutic treatments for large subsets of the users—such treatments being on par with pharmaceuticals in terms of their effectiveness in the general population. Accordingly, the therapeutic treatments developed in this manner can be delivered to patients without the need for individualized sensor measurements of, e.g., brain state and brain activity. This approach solves the problem of generalizability of treatment and results in reduced cost and other efficiencies in terms of the practical logistics of delivering therapeutic treatment.

Overall, the digital therapeutic has a huge market potential as it provides a non-invasive, cost-effective, and personalized method for treating AMD and other retinal degeneration disorders.

In disclosed embodiments, a method to treat eye disorders, such as retinal degeneration disorders, includes training a neural network (e.g., a generative neural network) to optimize computer-based acuity training to target specific neural circuits of the visual cortex to treat retinal degeneration disorders. This may include collecting a dataset of patients with different types of retinal degeneration disorders. The dataset may include information which characterizes the patient's visual deficits, such as visual acuity, contrast sensitivity, and visual field defects. The method further includes performing an acuity training program that involves presenting visual stimuli (e.g., semantic symbols or characters and/or other figurative images) to the patient through a display device. The visual stimuli can be presented in a variety of formats, such as videos, animations, and games and/or may be overlaid on such content. The method further includes creating a neural network architecture to optimize the acuity training program for each patient based on their unique visual deficits. The neural network is trained to identify the optimal parameters for each patient, such as the size, spacing, orientation, contrast, brightness, and/or color of the visual stimuli. The neural network may be trained using the dataset of patients with different types of retinal degeneration disorders or may focus on a specific disorder. The network is trained to identify the relationship between the patient's visual deficits and the optimal acuity training parameters. The neural network may be validated by testing it on a validation dataset of patients with retinal degeneration disorders (or the specific disorder) and/or a dataset of patients with normal vision. The performance of the neural network may be compared to standard acuity training programs to assess its effectiveness.

### BRIEF DESCRIPTION OF THE DRAWINGS

FIG. 1 depicts an embodiment of a system to generate a visual stimulus, using brain activity data measured while dynamic visual neuromodulatory codes are displayed to a participant in a target state and a current state, to produce physiological responses having therapeutic or performance-enhancing effects.

FIG. 2 depicts an embodiment of a method, usable with the system of FIG. 1, to generate a visual stimulus to produce physiological responses having therapeutic or performance-enhancing effects.

FIG. 3 depicts an embodiment of a system to deliver visual neuromodulatory codes generated with closed-loop approach using an optimized descriptive space.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts). Reference throughout this specification to “one implementation” or “an implementation” or “particular implementations” means that a particular feature, structure or characteristic described in connection with the implementation is included in at least one implementation. Thus, the appearances of the phrases “in one implementation” or “in an implementation” or “particular implementations” in various places throughout this specification are not necessarily all referring to the same implementation. Furthermore, the particular features, structures, or characteristics may be combined in any suitable manner in one or more implementations.

As used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise. The headings and Abstract of the Disclosure provided herein are for convenience only and do not interpret the scope or meaning of the implementations.

Physiology is a branch of biology that deals with the functions and activities of life or of living matter (e.g., organs, tissues, or cells) and of the physical and chemical phenomena involved. It includes the various organic processes and phenomena of an organism and any of its parts and any particular bodily process. Hence, the term “physiological” is used herein to broadly mean characteristic of or appropriate to the functioning of an organism, including human physiology. The term includes the characteristics and functioning of the nervous system, the brain, and all other bodily functions and systems.

The term “neurophysiology” refers to the physiology of the nervous system. The term “neural” and the prefix “neuro” likewise refer to the nervous system. As used herein, all of these terms and prefixes refer to the physiology of the nervous system and brain. In some instances, these terms and prefixes are used herein to refer to physiology more generally, including the nervous system, the brain, and physiological systems which are physically and functionally related to the nervous system and the brain.

The visual cortex processes visual information from the retina in a topographic manner, meaning that different regions of the cortex are specialized for processing different regions of the visual field. The part of the visual cortex responsible for processing peripheral vision is located in the more posterior regions of the cortex, while the part of the cortex responsible for processing central vision is located in the more anterior regions of the cortex. Visual information is conveyed from the retina via the optic nerves to the lateral geniculate nucleus of the thalamus. From the thalamus the optic radiations convey visual information to the visual cortex. Both the thalamus and the visual cortex contain several subregions that are retinotopically segregated to allow distinct processing of visual information from various parts of the visual field.

Peripheral vision and central vision are processed differently in the visual cortex due to differences in the properties of the retinal ganglion cells that project to these areas. The ganglion cells in the peripheral retina have larger receptive fields and are more sensitive to changes in contrast and motion, while the ganglion cells in the central retina have smaller receptive fields and are more sensitive to details, such as edges and textures. The visual cortex processes peripheral vision differently than central vision by using specialized areas of the cortex that are sensitive to the properties of the retinal ganglion cells that project to these areas.

The retinal circuit performs low-level visual processing, the initial stage in the analysis of visual images. It extracts from the raw images in the left and right eyes certain spatial and temporal features and conveys them to higher visual centers. The rules of this processing are very plastic. In particular, the retina must adjust its sensitivity to ever-changing conditions of illumination. This adaptation allows our vision to remain more or less stable despite the vast range of light intensities encountered during the course of each day.

Various aspects of dynamic visual information are also processed in a distributed manner across other cortical regions in the primate brain. For example, the middle temporal (MT) and medial superior temporal (MST) areas are specialized regions of the visual cortex that play an important role in processing visual information related to motion and perception of depth. These areas are located in the dorsal visual stream, which is responsible for the processing of visual information related to the spatial location and motion of objects.

The MT area, also known as V5, is a region of the visual cortex that is specialized for the processing of visual motion. It receives input from the primary visual cortex (V1) and processes information related to the direction, speed, and trajectory of visual stimuli. The MT area is involved in a variety of visual tasks, including tracking moving objects, perceiving motion-defined shapes, and discriminating between the motion of different objects.

The MST area is located adjacent to the MT area and is involved in processing visual information related to the perception of depth and the movement of objects in three-dimensional space. The MST area receives input from the MT area and integrates information related to visual motion, binocular disparity, and optic flow. The MST area is involved in a variety of visual tasks, including depth perception, visual navigation, and the perception of self-motion. The MT and MST areas are important for the processing of visual information related to motion and perception of depth. Dysfunction of these areas can lead to visual deficits, such as impaired motion perception and depth perception.

The middle temporal (MT) and medial superior temporal (MST) areas, which are located in the dorsal visual stream, are specialized for processing visual motion and are important for processing peripheral vision. The MT and MST areas receive input from the primary visual cortex (V1) and integrate information related to the direction, speed, and trajectory of visual stimuli. In contrast, the part of the visual cortex responsible for processing central vision is located in the more anterior regions of the cortex, including the primary visual cortex (V1) and the visual association areas. These areas are specialized for processing details, such as edges, textures, and colors, and are important for processing central vision.

Particular embodiments may include the use of stimulation in multiple zones in the periphery of the retina simultaneously with synchronous and asynchronous harmony and similar or disparate image sets to enable retraining and/or hyper training of active neural areas. In some cases, by using a combination of image parameters, such as angle, color, motion, brightness, image type (e.g., scene or face), or other constructs that are in the periphery, i.e., eccentric to the scotoma (lesion), the disclosed approaches can bolster functionality to create improved function. The parameters can be applied in a specific region or regions, such as circumferential regions formed of eccentric rings, that further enhance the ability of the periphery to recognize visual detail for better function and cortical interpretation. In normal physiology, most scene reconstruction occurs in the fovea (i.e., within 1-2 degrees of peripheral). By training the remaining region peripheral or eccentric to the scotoma, there is the ability to engage circuits to fulfill the function of the near periphery and thus enhance function.

Age-related macular degeneration (AMD) is a condition that affects the macula, the part of the retina responsible for central vision. AMD can cause irreversible damage to the macula, leading to a loss of central vision. However, the peripheral vision remains intact in most cases. Producing modulation in the part of the visual cortex responsible for peripheral vision can help patients with AMD by improving the processing of visual information from the intact peripheral visual field.

Computer-based visual therapy involves the use of specialized software programs and hardware devices to present visual stimuli to the patient. The stimuli are designed to target specific areas of the retina and visual cortex, and can be customized based on the patient's individual needs and goals. There are several approaches to computer-based visual therapy. Visual acuity training uses a series of exercises and games to help improve visual acuity by training the eyes to recognize and interpret visual information more effectively. Contrast sensitivity training involves improving the ability to see objects in low-contrast environments, which is often impaired in individuals with retinal degeneration. Perimetry training involves improving the patient's ability to see objects in their peripheral vision, which can be particularly important for those with conditions that affect central vision, such as AMD. Motion detection training involves improving the ability to detect and interpret motion, which can be challenging for individuals with retinal degeneration.

The visual cortex is the part of the brain responsible for processing visual information. It is organized into multiple areas that are specialized for processing different aspects of visual information. The primary visual cortex (V1) is the first cortical area that receives input from the retina. The visual cortex also includes specialized areas that are responsible for processing peripheral vision, such as the middle temporal area (MT) and the medial superior temporal area (MST).

In disclosed embodiments, modulation of the visual cortex using dynamic neuromodulatory codes can modulate the activity of the neural circuits in the visual cortex, leading to changes in visual function. The display and viewing of dynamic neuromodulatory codes is a non-invasive technique that produces neuromodulatory responses in the brain, e.g., in the visual cortex. The treatment targets the part of the visual cortex responsible for processing peripheral vision, such as the MT and MST areas. By modulating the activity of these areas, the techniques disclosed herein can improve the processing of visual information from the intact peripheral visual field, leading to improved visual function. Producing a modulatory effect on the visual cortex circuits responsible for peripheral vision can help patients with AMD optimize, enhance, and/or recover function by improving the processing of visual information from the intact peripheral visual field. Thus, driving neurostimulatory activity helps to, in effect, retrain parts of the visual cortex for adaptation. The treatment involves using dynamic neuromodulatory codes to modulate the activity of the neural circuits in the visual cortex, leading to changes in visual function. The treatment is non-invasive and has the potential to improve the quality of life of patients with AMD.

Disclosed embodiments provide a method of treatment for age-related macular degeneration involving producing modulation in the part of the visual cortex responsible for peripheral vision. The treatment involves using dynamic neuromodulatory codes to produce modulation in the part of the visual cortex responsible for peripheral vision. This is a non-invasive technique that uses dynamic neuromodulatory codes (DNC) displayed to a user to stimulate the brain. The treatment is designed to activate the neural circuits in the visual cortex that are responsible for processing peripheral vision. The treatment is delivered using, e.g., a mobile device that is capable of displaying one or more DNC, such as in the form of a video or an overlay over other displayed content.

The use of mobile devices to deliver dynamic neuromodulatory images has several potential advantages as a treatment for AMD. Mobile devices are widely accessible and can be used in the patient's home, reducing the need for frequent visits to a clinic. The treatment is non-invasive and can be easily customized to the patient's needs and preferences.

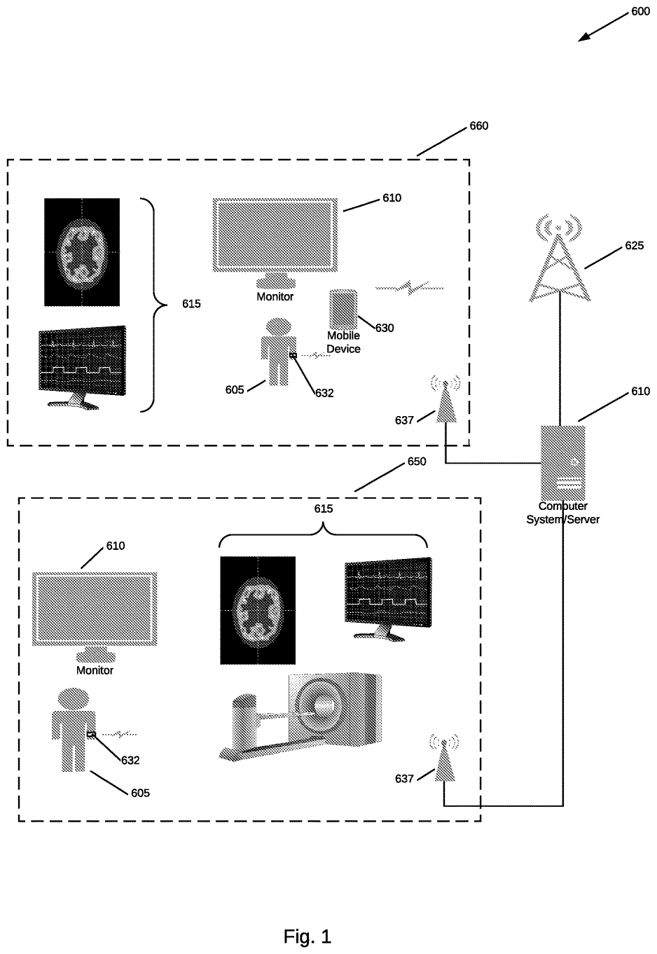

FIG. 1 depicts an embodiment of a system 600 to generate a visual stimulus, using brain activity data measured while dynamic visual neuromodulatory codes are displayed to a participant 605 in a target state and a current state, to produce physiological responses having therapeutic or performance-enhancing effects, as well as effects which enhance recovery and rehabilitation. The system 600 is processor-based and may include a network-connected computer system/server 610, or other type of computer system, having at least one processor and memory/storage. The memory/storage stores processor-executable instructions and data which, when executed by the at least one processor, cause the at least one processor to perform the necessary functions for the system to generate and provide to the user the visual stimulus.

In particular implementations, the computer system/server 610 is connected via a network 625 to a number of personal electronic devices 630, such as mobile phones and tablets, and computer systems. A dynamic visual neuromodulatory code may be generated based on feedback from one or more users and used as the visual stimulus to produce physiological responses having therapeutic or performance-enhancing effects, as discussed above.

The system 600 receives a first set of brain activity data measured, e.g., using a first test set up 650 including a display 610 and various types of brain state and/or brain activity measurement equipment 615, while a test participant 605 is in a target state, e.g., a state characterized by neuromodulatory stimulation of portion of the visual cortex responsible for peripheral vision. The target state may be induced in the participant 605 by providing known stimulus or stimuli, which may be in the form of dynamic visual neuromodulatory codes, as discussed above, and/or various other forms of stimulus, e.g., visual, video, chemical, and physical, etc.

The first set of brain state/activity data, thus, serves as a reference against which other measured sets of brain/activity can be compared to assess the effectiveness of a particular visual stimulus in achieving a desired state. The brain activity data may include, inter alia, data acquired from one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS)—measured while the participant is present in a facility equipped to make such measurements (e.g., a facility equipped with the first test set up 650). Various other types of physiological and/or neurological measurements may be used. Measurements of this type may be done in conjunction with an induced target state, as the participant will likely be present in the facility for a limited time.

The system further displays to the participant 605, using an electronic display 610, a candidate dynamic visual neuromodulatory code while the participant 605 is in a current state, the current state being different than the target state. For example, the participant 605 may be exhibiting limited stimulation of neural circuits in areas of the visual cortex responsible for central vision in a current state, as opposed to a state characterized by neuromodulatory stimulation of portion of the visual cortex responsible for peripheral vision in the target state. In particular implementations, the candidate dynamic visual neuromodulatory code may be based at least in part on or more initial dynamic visual neuromodulatory codes which are iteratively generated based at least in part on received feedback data indicative of responses of a group of participants during displaying of the one or more initial dynamic visual neuromodulatory codes to the group of participants.

The system 600 receives a second set of brain activity data measured, e.g., using a second test set up 660 including a display 610 and various types of brain state and/or brain activity measurement equipment 615, during the display of the candidate dynamic visual neuromodulatory code to the participant 605. As above, the brain activity data may include, inter alia, data acquired from one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS). The brain imaging may include functional imaging (see examples above) and/or structural imaging, e.g., MRI, etc. In particular implementations, both the first and the second sets of brain activity data may be obtained using the same test set up, i.e., either the first test set up 650 or the second test set up 660.

The system 600 performs an analysis the first set of brain state/activity data, i.e., the target state data, and the second set of brain state/activity data to produce at least one parameter indicative of an effectiveness of the candidate dynamic visual neuromodulatory code with respect to the participant 605. For example, feedback may be obtained from the participant 605 via administration one or more standard vision tests, e.g., a visual acuity test, contrast sensitivity test, and visual field test, during the target state (i.e., the desired state) and during the current state. In addition, various types of measured feedback data may be obtained (i.e., in addition to the imaging data mentioned above) while the participant 605 is in the target and/or current state. Analysis of such information can provide parameters and/or statistics indicative of an effectiveness of the candidate dynamic visual neuromodulatory code with respect to the participant.

Based at least in part on the parameters and/or statistics indicative of the effectiveness of the candidate dynamic visual neuromodulatory code, the system 600 outputs the candidate dynamic visual neuromodulatory code as the visual stimulus or performs a further iteration. In the latter case, the candidate dynamic visual neuromodulatory code is perturbed (i.e., algorithmically modified, adjusted, adapted, randomized, etc.). In particular implementations, the perturbing of the candidate dynamic visual neuromodulatory code may be performed using a machine learning model, a neural network, a convolutional neural network, a deep feedforward artificial neural network, an adversarial neural network, and/or an ensemble of neural networks. The displaying of the candidate dynamic visual neuromodulatory code to the participant is repeated and the system receives a further set of brain activity data measured during the displaying of the candidate dynamic visual neuromodulatory code. Analysis is again performed to determine whether to output candidate dynamic visual neuromodulatory code as the visual stimulus or to perform a further iteration.

In particular implementations, the system may generate a candidate dynamic visual neuromodulatory code from a set of “base” dynamic visual neuromodulatory codes. In such a case, the system iteratively generates base dynamic visual neuromodulatory codes having randomized characteristics, such as texture, color, geometry, etc. Neural responses to the base dynamic visual neuromodulatory codes are obtained and analyzed. For example, the codes may be displayed to a participant, or group of participants, with feedback data such as functional magnetic resonance imaging (fMRI) data, being obtained. Based at least in part on the result of the analysis of the neural responses to the base dynamic visual neuromodulatory codes, the system outputs a base dynamic visual neuromodulatory code as the candidate dynamic visual neuromodulatory code or perturbs one or more of the base dynamic visual neuromodulatory codes and performs a further iteration. In particular implementations, the perturbing of the base dynamic visual neuromodulatory codes may be performed using at is performed using at least one of: a machine learning model, a neural network, a convolutional neural network, a deep feedforward artificial neural network, an adversarial neural network, and an ensemble of neural networks.

In disclosed embodiments, a treatment protocol using DNC may involve patient selection and evaluation, in which case patients with AMD who have intact peripheral vision are suitable candidates for this treatment. The patient's medical history and visual function are evaluated to determine eligibility for the treatment.

Prior to treatment, high-resolution diagnostic imaging, such as with functional Magnetic Resonance Imaging (fMRI) scanning of the patient's brain coupled with visual stimuli presentation may be obtained. The fMRI scan is used to map the part of the visual cortex responsible for peripheral vision.

DNC which are specifically adapted to produce a neuromodulatory response in the part of the visual cortex responsible for peripheral vision are delivered to a patient, e.g., by displaying the DNC on a mobile device or other screen device. The DNC is adapted to target a specific area of the brain identified in the fMRI mapping. In embodiments, a peripheral test may be performed, e.g., on a smartphone screen, to determine the extent of the central vision loss as well to identify the areas where visual training should be concentrated. For example, the identification of such areas can be informed by having the patient tapping areas where they can see clearly on the screen.

In embodiments, the treatment may be delivered over a period of several weeks, or months, with multiple sessions per week. The frequency and duration of the treatment may be determined based on the patient's response to the treatment. The patient's visual function may be monitored throughout the treatment using standard visual function tests. The progress of the treatment is evaluated based on the improvement in visual function. The treatment is non-invasive, meaning that it does not require any surgical procedures. The treatment is adapted to improve visual function in patients with AMD, particularly in the improvement of the patient's ability to navigate in the environment.

In view of the above, modulation of the visual cortex using DNC represents a promising treatment for age-related macular degeneration. The treatment targets the part of the visual cortex responsible for peripheral vision and has the potential to improve the visual function in patients with this condition.

In embodiments, dynamic visual stimuli can be used to train the patient's peripheral vision to perform functions normally performed by the central vision. This training involves presenting dynamic visual stimuli, e.g., DNC, that are designed to stimulate the neural circuits in the visual cortex responsible for peripheral vision, encouraging the brain to adapt and reorganize to perform tasks normally performed by the central vision.

Embodiments may include training of eccentric viewing. AMD can lead to a central scotoma, which is a loss of central vision. Individuals with AMD can learn to use their peripheral vision, also known as eccentric viewing, to compensate for the loss of central vision. DNC-based vision therapy provides dynamic neuromodulatory codes (DNC) adapted to stimulate the neural circuits in the visual cortex responsible for peripheral vision, which may result in greater use of eccentric viewing, thereby improving a patient's visual function.

AMD can lead to a slower processing speed for visual information, which can make it difficult to process visual information quickly. DNC-based vision therapy can provide training that provides dynamic neuromodulatory codes (DNC) adapted to stimulate the neural circuits in the visual cortex responsible for peripheral vision in conjunction with exercises that improve visual processing speed, such as visual search tasks and reaction time exercises. AMD can have a significant impact on an individual's quality of life. DNC-based vision therapy can provide vision-related quality of life assessments that measure the impact of AMD on an individual's daily activities, such as reading, driving, and socializing.

DNC-based vision therapy can be performed at home, which provides convenience for individuals with AMD. Home-based treatment allows individuals to perform visual exercises on their own schedule and in a comfortable environment.

In embodiments, a training protocol or method may include patient selection and evaluation, in which patients with age-related macular degeneration or other conditions that affect the macula are evaluated to determine the extent of the visual impairment and the eligibility for the treatment. The method may further include the generation of dynamic visual stimuli, e.g., in the form of dynamic neuromodulatory codes (DNC), to stimulate the neural circuits in the visual cortex responsible for peripheral vision. The images are adapted to provide the greatest efficacy in stimulating the targeted regions of the visual cortex, as described in further detail below.

The method may further include peripheral vision training, in which a patient is presented with the DNC while performing tasks that are normally performed by the central vision, such as reading, recognizing faces, or navigating through a virtual environment. The stimuli are designed to encourage the patient to use their peripheral vision to perform these tasks. The method may further include gradual adaptation, in which the training protocol is adapted to gradually increase the complexity and difficulty of the tasks, encouraging the patient to rely more on their peripheral vision to perform them. The method may further include monitoring of progress, in which the patient's visual function is monitored throughout the training using standard visual function tests. The progress of the training protocol or method may be evaluated based on the improvement in visual function experienced by the patient.

The use of dynamic visual stimuli to train the patient's peripheral vision to perform functions normally performed by the central vision has several potential advantages as a treatment for AMD. The training can promote neural plasticity (i.e., neurofunctional efficacy by adaptive training of existing circuits resulting in neurofunctional enhancement) and adaptation in the visual cortex, preventing visual cortical atrophy and encouraging the brain to reorganize and compensate for the loss of central vision. The training can also be customized to the individual needs and preferences of the patient.

Modulation of the portion of the visual cortex responsible for peripheral vision is a promising treatment for age-related macular degeneration (AMD). The treatment involves using DNC to modulate the activity of the neural circuits in the visual cortex, leading to changes in visual function. The following are aspects of performing modulation of the portion of the visual cortex responsible for peripheral vision as a treatment for AMD. In one aspect, there may be patient selection and evaluation in which patients with AMD who have intact peripheral vision are considered to be suitable candidates for this treatment. The patient's medical history and visual function may be evaluated to determine eligibility for the treatment. In another aspect, there may be imaging and mapping of the patient's brain, e.g., using functional Magnetic Resonance Imaging (fMRI). Prior to treatment, a high-resolution MRI scan of the patient's brain is obtained. The fMRI scan is used to map the part of the visual cortex responsible for peripheral vision.

Based on the mapping, DNC may be generated and/or selected which are adapted to produce a neuromodulatory response in the portion of the visual cortex responsible for peripheral vision. The DNC may be adapted based on the individual patient's neurophysiology and/or may be based on neurophysiological measurements performed on a population of subjects to produce a generalized DNC library.

The treatment may be delivered over a period of several weeks, with multiple sessions per week. The frequency and duration of the treatment may be determined based at least in part on the patient's response to the treatment. The patient's visual function may be monitored throughout the treatment using standard visual function tests. The progress of the treatment is evaluated based on the improvement in visual function. After the initial treatment period, maintenance sessions may be needed to maintain the treatment's effect. The frequency and duration of these sessions may be determined based at least in part on the patient's response to the initial treatment.

In embodiments, modulation of the portion of the visual cortex responsible for peripheral vision may be used in conjunction with neural feedback as a treatment for AMD.

This treatment approach involves using a combination of DNC and neural feedback to improve the processing of visual information from the intact peripheral visual field. As above, there may be patient selection and evaluation, including evaluation of the patient's medical history and visual function to determine eligibility for the treatment. Prior to treatment, a high-resolution MRI scan of the patient's brain may be obtained to map the part of the visual cortex responsible for peripheral vision.

DNC may be delivered, e.g., via display on a mobile device screen, to the patient which are specifically adapted to produce modulation in the portion of the visual cortex responsible for peripheral vision. Neural feedback may be used to provide real-time feedback on the patient's neuromodulatory response and their visual processing. This feedback can be provided using a variety of techniques, such as electroencephalography (EEG), magnetoencephalography (MEG) or functional magnetic resonance imaging (fMRI). The treatment may be delivered over a period of several weeks, with multiple sessions per week. The frequency and duration of the treatment may be determined based at least in part on the patient's response to the treatment. The patient's visual function may be monitored throughout the treatment using standard visual function tests. The progress of the treatment may be evaluated based on the improvement in visual function.

The combination of DNC delivery and neural feedback has several potential benefits as a treatment for AMD. DNC can modulate the activity of the neural circuits in the visual cortex, leading to changes in visual function. Neural feedback can provide real-time feedback on the patient's neurophysiology and visual processing, which can enhance the efficacy of the treatment. Measuring a subject's neuromodulatory response to dynamic visual stimuli and using this response as feedback to optimize the stimuli may involve using various imaging modalities, such as functional magnetic resonance imaging (fMRI), electroencephalography (EEG), or magnetoencephalography (MEG), to monitor the subject's brain activity while viewing the dynamic visual stimuli.

A treatment protocol or method may be described as follows. In the subject selection and evaluation, subjects with age-related macular degeneration or other conditions that affect the macula may be evaluated to determine the extent of the visual impairment and the eligibility for the treatment. Dynamic visual stimuli, e.g., DNC, are created using computer algorithms that generate visual stimuli designed to produce a neuromodulatory response in the neural circuits in the visual cortex responsible for peripheral vision. The DNC are adapted to provide the greatest efficacy in stimulating the targeted regions of the visual cortex. The subject's neural activity may be monitored while viewing the dynamic visual stimuli using various imaging modalities, such as fMRI, EEG, or MEG. These techniques can provide real-time feedback about the neural activity in the visual cortex, allowing for the optimization of the dynamic visual stimuli.

The feedback obtained from the imaging modalities is used to optimize the dynamic visual stimuli. This process may involve iteratively adjusting the visual stimuli, or modifying the content of the stimuli to enhance their efficacy. The use of neural feedback to optimize dynamic visual stimuli has several potential advantages as a treatment for age-related macular degeneration. The feedback can provide real-time information about the neural activity in the visual cortex, allowing for the optimization of the stimuli to provide the greatest efficacy. This approach can also be customized to the individual needs and preferences of the subject.

Dynamic visual stimuli, such as dynamic neuromodulatory codes (DNC), can be generated and adapted using a high-resolution neuroimaging system, such as a functional magnetic resonance imaging (fMRI) or magnetoencephalography (MEG) system. These systems provide real-time information about the neural activity in the visual cortex, allowing for the creation and optimization of visual stimuli that are tailored to the individual needs and preferences of the subject. The DNC may be optimized to provide the greatest efficacy in stimulating neuromodulatory responses in the targeted regions of the visual cortex. The subject's neural activity may be monitored while viewing the DNC using a high-resolution neuroimaging system, such as fMRI or MEG, during the generation of the DNC to adapt the DNC (e.g., in the form of video images) to particular applications or during use to obtain performance optimization.

As discussed above, age-related macular degeneration (AMD) is a condition that affects the macula, the part of the retina responsible for central vision. The disease can cause irreversible damage to the macula, leading to a loss of central vision. The use of DNC-based vision therapy in combination with drugs can help slow the progression of the disease and improve visual function. Drug therapy is typically used to slow the progression of the wet form of the disease and to preserve visual function. The drugs commonly used to treat AMD are anti-vascular endothelial growth factor (VEGF) agents, such as bevacizumab, ranibizumab, and aflibercept. These drugs are injected into the eye and can reduce the growth of abnormal blood vessels and leakage of fluid in the macula that leads to wet AMD.

DNC-based vision therapy is used to improve the processing of visual information in the intact peripheral visual field. The therapy involves providing dynamic neuromodulatory codes (DNC) adapted to stimulate the neural circuits in the visual cortex responsible for peripheral vision. The therapy can improve visual function in the intact peripheral visual field, which can compensate for the loss of central vision caused by the disease.

The combination of DNC-based vision therapy and drug therapy can help slow the progression of AMD and improve visual function. The drugs can reduce the growth of abnormal blood vessels and leakage of fluid in the macula, while the DNC-based vision therapy can improve the processing of visual information in the intact peripheral visual field, which can compensate for the loss of central vision caused by the disease.

In addition to drugs aimed at reducing progressive retinal deterioration another embodiment includes the combination of visual DNC-based vision therapy with drugs that enhance visual cortical function. For example, the local circuitry of the visual cortex involved in peripheral vision uses acetylcholine (ACh) neurotransmission via muscarinic ACh receptors, which could be enhanced through the use of acetylcholine esterase inhibitors (AChI) such as Donepezil or muscarinic agonists such as arecoline. The combination of such pharmacological and DNC-based neurophysiological intervention to enhance peripheral visual attention is envisioned here as a powerful way to improve ramining vision and compensate for lost vision. Other therapeutic combinations of DNC-based vision therapy with drugs acting on the glutamate and gamma-amino butyric acid (GABA) neurotransmission are also envisioned.

Disclosed embodiments provide a non-invasive and cost-effective method for treating AMD. The method involves using dynamic visual neuromodulatory stimulation produced by the screen of a digital device to stimulate neuromodulatory response in the visual cortex to improve visual function. This may involve an assessment of visual function, before initiating the treatment, in which the patient's visual function is assessed using standard tests, such as the visual acuity test, contrast sensitivity test, and visual field test. The results of these tests may be used to determine the severity of the disease and to monitor the progress of the treatment.

Based at least in part on the assessment of visual function, a customized visual neuromodulatory stimulation program may be designed for each patient. The program will be designed to stimulate neuromodulatory response in specific portions of the visual cortex, e.g., the portions associated with peripheral vision, by presenting DNC on the screen of a digital device, e.g., a user's mobile device.

In implementations, the patient may be instructed to perform the visual neuromodulatory stimulation program daily for a prescribed duration of time, typically 20-30 minutes per session. The patient will use a digital device, such as a tablet or smartphone, to perform the visual neuromodulatory stimulation program, which includes displaying one or more DNC on the device screen, either alone or superimposed with other displayed material. The patient's visual function may be monitored periodically during the treatment to assess the efficacy of the visual neuromodulatory stimulation program. The visual function tests may be repeated periodically to monitor the progress of the treatment.

Among other advantages, embodiments disclosed herein provide a non-invasive and cost-effective method for treating age-related macular degeneration (AMD). The method is easy to administer and does not require the use of invasive procedures. The method can be performed at home, making it convenient for patients. The method can also be customized for each patient, providing individualized treatment.

Dynamic neuromodulatory images, such as video, can be used to effect a targeted neuromodulatory response in the visual cortex of a patient. This is achieved by presenting visual neuromodulatory stimuli designed to stimulate responses in specific regions of the visual cortex responsible for peripheral vision, using a mobile device such as a smartphone or tablet. Dynamic neuromodulatory images may be created by using computer algorithms that generate visual neuromodulatory stimuli designed to stimulate the neural circuits in the visual cortex responsible for peripheral vision. The images are adapted to provide the greatest efficacy in stimulating the targeted regions of the visual cortex.

The dynamic neuromodulatory images are presented to the patient using a mobile device, such as a smartphone or tablet. The patient views the images for a prescribed duration and frequency, as determined by the treatment protocol or method. The patient's visual function may be monitored throughout the treatment using standard visual function tests. The progress of the treatment may be evaluated based at least in part on the improvement in visual function.

The dynamic neuromodulatory images can be presented to the patient in a variety of formats, including videos, animations, or games. The visual stimuli are designed to engage the neural circuits in the visual cortex responsible for peripheral vision, promoting plasticity (i.e., neurofunctional efficacy by adaptive training of existing circuits resulting in neurofunctional enhancement) and adaptation in these circuits.

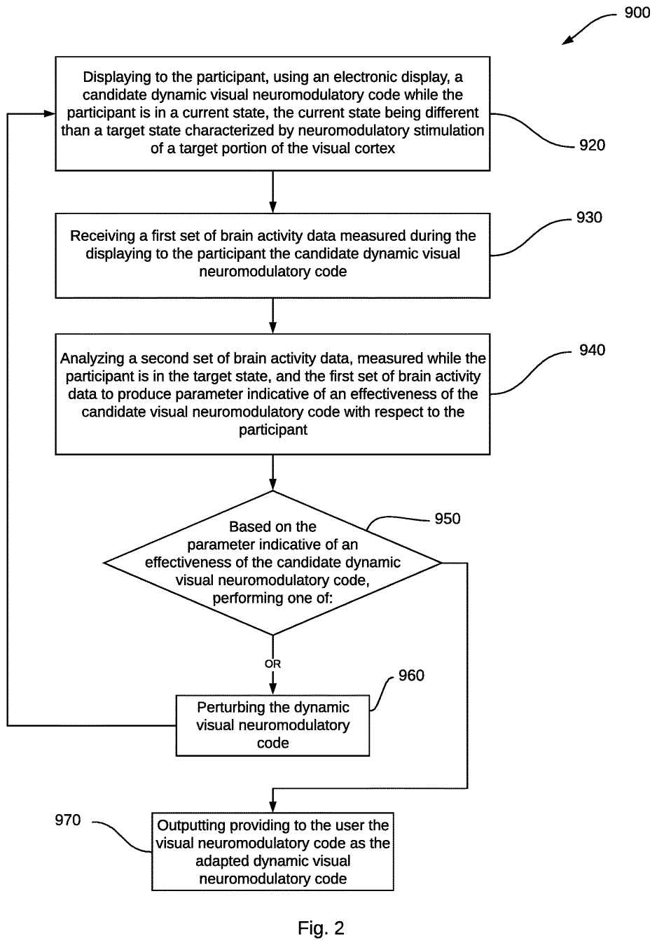

FIG. 2 depicts an embodiment of a method 900 to generate and provide to a user a visual stimulus to produce physiological responses having therapeutic or performance-enhancing effects. The disclosed method is usable in a system such as that shown in FIG. 1, which is described above.

The method 900 includes displaying to the participant (using an electronic display) a candidate dynamic visual neuromodulatory code while the participant is in a current state, the current state being different than a target state characterized by neuromodulatory stimulation of a target portion of the visual cortex (920). The method 900 further includes receiving a first set of brain activity data measured during the displaying to the participant the candidate dynamic visual neuromodulatory code (930). The method 900 further includes analyzing a second set of brain activity data, measured while the participant is in the target state, and the first set of brain activity data to produce at least one parameter indicative of an effectiveness of the candidate dynamic visual neuromodulatory code with respect to the participant (940).

Based at least in part on the at least one parameter indicative of an effectiveness of the candidate dynamic visual neuromodulatory code, the method further includes performing (950) one of: (i) outputting the candidate dynamic visual neuromodulatory code as the visual stimulus (970), and (ii) perturbing the candidate dynamic visual neuromodulatory code and repeating the displaying to the participant the candidate dynamic visual neuromodulatory code, the receiving the first set of brain activity data measured during the displaying to the participant the candidate dynamic visual neuromodulatory code, and the analyzing the second set of brain activity data and the first set of brain activity data (960).

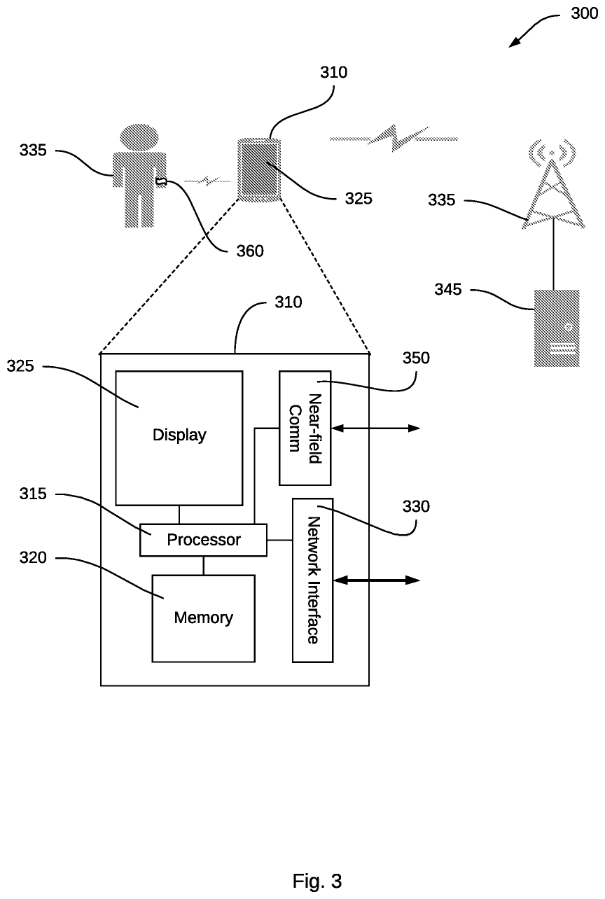

FIG. 3 depicts an embodiment of a system 300 to deliver visual neuromodulatory codes generated with closed-loop approach using an optimized descriptive space. The system 300 includes an electronic device, referred to herein as a user device 310, such as mobile device (e.g., mobile phone or tablet) or a virtual reality headset. A patient views the visual neuromodulatory codes on a user device, e.g., a smartphone or tablet, using an app or by streaming from a website. In embodiments, the app or web-based software may provide for the therapeutic visual neuromodulatory codes to be merged with (e.g., overlaid on) content being displayed on the screen, e.g., a website being displayed by a browser, a user interface of an app, or the user interface of the device itself, without interfering with normal use of such content. Thus, disclosed embodiments provide functionality akin to a dynamic lens or filter between the content to be displayed and the viewer.

In embodiments, the system may be adapted to personalize the visual neuromodulatory codes through the use of sensors and data from the user device (e.g., smartphone). For example, the user device may provide for eye-tracking, and pupil dilation measurement using a camera of the user device. Furthermore, the user device may present vision-related tests and/or questionnaires to a patent, developed using artificial intelligence, to automatically individualize the visual neuromodulatory codes and exposure time for optimal therapeutic effect.

The user device 310 comprises at least one processor 315 and memory 1420 (e.g., random access memory, read-only memory, flash memory, etc.). The memory 320 includes a non-transitory processor-readable medium adapted to store processor-executable instructions which, when executed by the processor 315, cause the processor 315 to perform a method to deliver the visual neuromodulatory codes. The user device 310 has an electronic display 325 adapted to display images rendered and output by the processor 315.

The user device 310 also has a network interface 330, which may be implemented as a hardware and/or software-based component, including wireless network communication capability, e.g., Wi-Fi or cellular network. The network interface 330 is used to retrieve one or more adapted visual neuromodulatory codes, which are adapted to produce physiological responses having therapeutic or performance-enhancing effects 335. In some cases, visual neuromodulatory codes may be retrieved in advance and stored in the memory 320 of the user device 310.

In implementations, the retrieval, e.g., via the network interface 330, of the adapted visual neuromodulatory codes may include communication via a network, e.g., a wireless network 340, with a server 345 which is configured as a computing platform having one or more processors, and memory to store data and program instructions to be executed by the one or more processors (the internal components of the server are not shown). The server 345, like the user device 310, includes a network interface, which may be implemented as a hardware and/or software-based component, such as a network interface controller or card (NIC), a local area network (LAN) adapter, or a physical network interface, etc. In implementations, the server 345 may provide a user interface for interacting with and controlling the retrieval of the visual neuromodulatory codes.

The processor 315 outputs, to the display 325, visual neuromodulatory codes adapted to produce physiological responses having therapeutic or performance-enhancing effects in a user 335 viewing the display 325. The visual neuromodulatory codes may be generated by any of the methods disclosed herein. In this manner, the visual neuromodulatory codes are presented to the user 335 so that the therapeutic or performance-enhancing effects can be realized. In outputting the adapted visual neuromodulatory codes to the display 325 of the user device 310, each displayed visual neuromodulatory code, or sequence of visual neuromodulatory codes (i.e., visual neuromodulatory codes displayed in a determined order), may be displayed for a determined time. These features provide, in effect, the capability of establishing a “dose” which can be prescribed for the user on an individualized basis, in a manner analogous to a prescription medication. In implementations, the determined display time of the adapted visual neuromodulatory codes may be adapted based on user feedback data indicative of responses of the user 335. In implementations, outputting the adapted visual neuromodulatory codes may include overlaying the visual neuromodulatory codes on displayable content, such as, for example, the displayable output of an app running on the user device, the displayable output of a browser running on the user device 310, and the user interface of the user device 310.

The user device 310 also has a near-field communication interface 350, e.g., Bluetooth, to communicate with devices in the vicinity of the user device 310, such as, for example, sensors (e.g., 360) to measure physiological responses of the subject 335 while the visual neuromodulatory codes are being presented to the subject 335. In implementations, the sensors may include components of the user device 310 itself, which may obtain feedback data by, e.g., tracking eye movement, and receiving input to displayed prompts.

As noted above, the app or web-based software running on the user device 310 may provide for the therapeutic visual neuromodulatory codes to be merged with (e.g., overlaid on) content being displayed on the screen, e.g., a website being displayed by a browser, a user interface of an app, or the user interface of the device itself, without interfering with normal use of such content. In embodiments, the user device 310 presents displayable content and adapted visual neuromodulatory codes on the display 325 in combination, thereby allowing a user to view displayable content, such as the output of an application or a webpage displayed by a web browser, while at the same time receiving treatment in the form of adapted visual neuromodulatory codes. This approach lessens the burden on the user, because the treatment is done while the user is attending to the ordinary functioning of the user device 310. Furthermore, because this approach can be integrated into an existing device, it allows for a user to receive treatment without acquiring a custom piece of hardware, i.e., a hardware device specifically designed for treatment.

In disclosed embodiments, the dynamic neuromodulatory codes may be in the form of semantic content, such as letters, numbers, etc., or other types of figurative images. In such a case, the semantic content may be adapted in a manner similar to the adaptation of non-figurative and/or non-semantic images, as disclosed herein. Furthermore, the semantic images may be used as a component of acuity tests and acuity training. This may include training a neural network, e.g., a generative neural network (GAN), with acuity training datasets to adapt the acuity training to achieve more effective and more targeted results. The training would be based on neural objectives, as discussed above with respect to dynamic neuromodulatory codes (DNC) and/or behavioral feedback, such as subject responses.

More generally, the neural objectives for acuity training using semantics could include neuronal activation time and/or strength, e.g., as measured using fMRI. In embodiments, a GAN could be trained so that it is optimized for speed of activation, encoded neural locations, and/or behavioral responses. In embodiments, a combination of DNC and semantic images may be used to produce target neuronal responses. In some cases, content, e.g., DNC in the form of video may be overlaid on other screen content using a “Content Lens” implementation, as discussed below.

The use of semantic and other types of figurative visual stimuli, alone or in combination with DNC-based stimulation, provides a generalizable acuity training and excitement of visual processing to improve vision generally. These approaches may adapt to the particular type of visual disease and the severity of disease based at least in part on measured neural response. Indeed, in particular embodiments, these approaches may be agnostic to type of disease and the severity level.

For example, instead of a relatively simple form of acuity training, such as a subject following dots displayed on a screen, the trained neural network may present permutations of geometric shapes, letters, numbers, characters, and/or symbols to maximize the effectiveness of acuity training. This is combinable with non-semantic approaches using DNC generated by processes disclosed herein. This approach to acuity training, and acuity testing, is more effective because a neural network optimizes the outputs of the acuity test, e.g., letters, dots, and/or numbers, to improve the acuity training, which, in turn, may improve and accelerate treatment. As such, any evaluation of therapy effectiveness based on acuity tests, e.g., by the Food & Drug Administration (FDA) would reflect these improved capabilities. The neural networks would also optimize parameters relating to acuity dynamics, such as speed, rotation, color, etc., of presented semantics. Presenting semantics, e.g., letters, controlled with such dynamic parameters provides an improved therapeutic effect over conventional acuity tests which have not been adapted to specific neural and/or behavioral objectives.

Following the retrieval or generation of adapted visual neuromodulatory codes (as described above), the adapted visual neuromodulatory codes may be combined with displayable content to form one or more dynamic neuromodulatory composite images. The combining of the adapted visual neuromodulatory codes with displayable content may include performing image overlay using techniques such as pixel addition, multiply blend, screen blend, and alpha compositing. Various other image overlay techniques may be employed. A particular overlay technique may be selected by subjectively evaluating the appearance of the dynamic composite image, e.g., its clarity, brightness, contrast, etc. An overlay technique may also be selected by comparing the effectiveness of the resulting dynamic neuromodulatory composite images on test subjects.

One example of an image overlay technique, alpha compositing (or “alpha blending), is the process of combining one image with a background to create the appearance of partial or full transparency. A color combination is stored for each image element (i.e., pixel), e.g., a combination of red, green, and blue. Each pixel also has an additional numeric value, a, with a value ranging from 0 to 1—referred to as an “alpha channel.” A value of 0 means that the pixel is fully transparent and the color in the pixel beneath will show through. A value of 1 means that the pixel is fully opaque.

With the existence of an alpha channel, it is possible to express compositing image operations using a compositing algebra. For example, given two images A and B, the most common compositing operation is to combine the images so that A appears in the foreground and B appears in the background. This is expressed as A over B. As an example, the over operator can be accomplished by applying the following formula to each pixel:

$${α}_{0} = {α}_{a} + {α}_{b} ( 1 - {α}_{a} )$$

$${c}_{0} = \frac{{C}_{a} {α}_{a} + {C}_{b} {α}_{b} ( 1 - {α}_{a} )}{{α}_{0}}$$

where C₀, Cₐ, and C_{b} stand for the color components of the pixels in the result, image A and image B, respectively, applied to each color channel (i.e., red/green/blue) individually, and a₀, aₐ, and a_{b} are the alpha values of the respective pixels. With “premultiplied alpha,” the RGB components are multiplied by their corresponding alpha values, thereby representing the emission of the object or pixel (with the alpha values representing the occlusion). In such a case, the color components become:

$${C}_{0} = {C}_{a} + {C}_{b} ( 1 - {α}_{a} )$$

The composite images are output to the display 325 by the processor 315. The displayable content may include such things as the displayable output of an application, a browser, and/or a user interface of the user device. Each of the dynamic neuromodulatory composite images may be displayed for a determined time period which may be adapted based on user feedback data (e.g., feedback data indicative of neurological and/or physiological responses of the user).

In embodiments, the retrieval or generation of adapted visual neuromodulatory codes and the combining of the adapted visual neuromodulatory codes with displayable content may be performed, at least in part, by a graphics processing unit (GPU) (not shown) of the user device 310, thereby allowing the processor 315 of the user device 310 to operate without being burdened by additional processing tasks.

The user device 310 may obtain user feedback data, e.g., feedback data which is indicative of neurological and/or physiological responses of the user, during the outputting of the dynamic neuromodulatory composite images to the electronic display 325. The user feedback data may be obtained, for example, using components of the user device 310 to track eye movement and/or receive input to displayed prompts. Various other types of components may be used to measure various types of user feedback data. In some cases, the user feedback data may be obtained by receiving data from a wearable neurological sensor.

In embodiments, output may be received from sensors that measure eye movements of the user during the outputting of the visual neuromodulatory codes by the user device 310. For example, a forward-facing camera of the user device 310 may be used as a sensor to track eye movements. In such a case, the processor 315 may execute software to analyze images and/or video taken by the forward-facing camera to identify positions and track movement of the user's eyes. Other types of sensors and measurement techniques may also be used to perform these functions. In some cases, hardware and software components of the user device 310 which perform facial recognition may perform, or assist in performing, the eye movement tracking.

Based on the analyzed output of the sensors that measure the eye movements of the user, a visual focal location of the user on the electronic display 325 may be determined. Values for a set of adapted rendering parameters may be calculated based on the determined visual focal location of the user on the electronic display 325. In such a case, the adapted rendering parameters may effectively shift one or more key reference locations of the displayed visual neuromodulatory codes to align with the determined visual focal location of the user to ensure that the user's attention is directed to the most effective portion. For example, the reference locations of the displayed visual neuromodulatory codes may be shifted to align with a visual focal location which moves across the screen as the user reads the displayable content.

The above embodiments involve the use of non-figurative (i.e., abstract, non-semantic, and/or non-representational) visual stimuli, such as the visual neuromodulatory codes described herein, which have advantages over figurative content. Non-figurative visual stimuli can be brought under tight experimental control for the purpose of stimulus optimization. Under AI guidance, specific features (e.g., shape, color, duration, movement, frequency, hue, etc.) can be expressed as parameters and gradually readjusted and recombined, frame by frame, pixel by pixel, to drive bio-response in the desired direction. Unlike pictures of people or scenes, non-figurative visual stimuli are free of cultural or language bias and thus more generalizable as a global therapeutic. Furthermore, non-figurative images are less likely to interfere with displayable content when combined as a composite image.

To activate specific targeted areas in the visual cortex, neuronal selectivity can be examined using the vast hypothesis space of a generative deep neural network, without assumptions about features or semantic categories. A genetic algorithm can be used to search this space for stimuli that maximize neuronal firing and/or feedback data indicative of responses of a user, or group of participants, during display of the stimuli. This allows for the evolution of synthetic images of objects with complex combinations of shapes, colors, and textures, sometimes resembling animals or familiar people, other times revealing novel patterns that do not map to any clear semantic category.

In embodiments, a combination of a pre-trained deep generative neural network and a genetic algorithm can be used to allow neuronal responses and/or feedback data indicative of responses of a user, or group of participants, during display of the stimuli to guide the evolution of synthetic images. By training large numbers of images, a generative adversarial network can learn to model the statistics of natural images without merely memorizing the training set, thus representing a vast and general image space constrained only by natural image statistics. This provides an efficient space in which to perform a genetic algorithm, because the brain also learns from real-world images, so its preferred images are also likely to follow natural image statistics.

Aspects of the presently taught approaches and techniques may be embodied in the form of a system, a computer program product, or a method. Similarly, aspects of the presently taught approaches and techniques may be embodied as hardware, software, or a combination of both. Aspects of the presently taught approaches and techniques may be embodied as a computer program product comprised in (e.g. saved on, conveyed by or the like) one or more computer-readable media in the form of computer-readable program code embodied thereon.

A computer-readable medium may be a computer-readable storage medium and/or a computer-readable transmission medium. A computer-readable storage medium (or non-transitory computer-readable medium) may be, for example, an electronic, optical, magnetic, electromagnetic, infrared, or semiconductor system, apparatus, or device, or any combination thereof. A computer readable transmission medium may include carrier waves, transmission signals or the like. A computer-readable transmission medium may convey instructions between components of a single computer system and/or between plural separate computer systems.

Computer program code in embodiments of the presently taught approaches and techniques may be written in any suitable programming language. The program code may execute on a single computer, or on a plurality of computers and/or processors. The computer may include a processing unit in communication with a computer-usable medium, where the computer-usable medium contains a set of instructions, and where the processing unit is designed to carry out the set of instructions.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. Those of skill in the art will recognize that many of the methods or algorithms set out herein may employ additional acts, may omit some acts, and/or may execute acts in a different order than specified. The various implementations described above can be combined to provide further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A method for treating eye disorders, comprising:
retrieving one or more adapted visual neuromodulatory codes, said one or more adapted visual neuromodulatory codes being adapted to produce neuromodulatory responses in the visual cortex; and
outputting to an electronic display of a device viewable by a patient said one or more adapted visual neuromodulatory codes.

2. The method of claim 1, wherein said one or more adapted visual neuromodulatory codes are adapted to improve processing of visual information in a target neural area.

3. The method of claim 1, wherein said one or more adapted visual neuromodulatory codes are adapted to improve processing of visual information in an intact peripheral visual field.

4. The method of claim 3, further comprising performing drug therapy to slow progression of a disorder and/or improve visual function, wherein the drug therapy is designed to reduce growth of abnormal blood vessels and leakage of fluid in the macula.

5. The method of claim 4, wherein wherein the drug therapy comprises anti-vascular endothelial growth factor (VEGF) agents.

6. The method of claim 1, wherein said one or more adapted visual neuromodulatory codes are generated by:
analyzing a first set of brain activity data to produce at least one parameter indicative of an effectiveness of a candidate dynamic visual neuromodulatory code with respect to a participant;
performing, based at least in part on the at least one parameter indicative of an effectiveness of the candidate dynamic visual neuromodulatory code, one of:
(i) outputting the candidate dynamic visual neuromodulatory code as an adapted dynamic visual neuromodulatory code; and
(ii) perturbing the candidate dynamic visual neuromodulatory code, displaying to the participant the perturbed candidate dynamic visual neuromodulatory code, receiving a second set of brain activity data measured during the displaying to the participant the perturbed candidate dynamic visual neuromodulatory code, and iteratively repeating said analyzing using the second set of brain activity data to produce at least one parameter indicative of an effectiveness of the candidate dynamic visual neuromodulatory code with respect to the participant.

7. The method of claim 6, wherein said perturbing comprises obtaining a further candidate dynamic visual neuromodulatory code from a machine learning model trained to target specific neural circuits of the visual cortex to treat retinal degeneration disorders.

8. The method of claim 7, wherein training of the machine learning model comprises
displaying dynamic visual neuromodulatory codes to subjects with different types of retinal degeneration disorders; and
measuring neural responses from the subjects during said displaying.

9-37. (canceled)

38. A system for treating eye disorders, comprising:
a device having one or more processors in communication with a memory, the memory storing instructions executable by said one or more processors to perform:
retrieving one or more adapted visual neuromodulatory codes, said one or more adapted visual neuromodulatory codes being adapted to produce neuromodulatory responses in the visual cortex; and
outputting to an electronic display of the device viewable by a patient said one or more adapted visual neuromodulatory codes.

39. The system of claim 38, wherein said one or more adapted visual neuromodulatory codes are adapted to improve processing of visual information in an intact peripheral visual field.

40. The system of claim 39, further comprising performing drug therapy to slow progression of a disorder and/or improve visual function, wherein the drug therapy is designed to reduce growth of abnormal blood vessels and leakage of fluid in the macula.

41. The system of claim 40, wherein the drug therapy comprises anti-vascular endothelial growth factor (VEGF) agents.

42. The system of claim 38, wherein said one or more adapted visual neuromodulatory codes are generated by:
analyzing a first set of brain activity data to produce at least one parameter indicative of an effectiveness of a candidate dynamic visual neuromodulatory code with respect to a participant;
performing, based at least in part on the at least one parameter indicative of an effectiveness of the candidate dynamic visual neuromodulatory code, one of:
(i) outputting the candidate dynamic visual neuromodulatory code as an adapted dynamic visual neuromodulatory code; and
(ii) perturbing the candidate dynamic visual neuromodulatory code, displaying to the participant the perturbed candidate dynamic visual neuromodulatory code, receiving a second set of brain activity data measured during the displaying to the participant the perturbed candidate dynamic visual neuromodulatory code, and iteratively repeating said analyzing using the second set of brain activity data to produce at least one parameter indicative of an effectiveness of the candidate dynamic visual neuromodulatory code with respect to the participant.

43. The system of claim 42, wherein said perturbing comprises obtaining a further candidate dynamic visual neuromodulatory code from a machine learning model trained to target specific neural circuits of the visual cortex to treat retinal degeneration disorders.

44. The system of claim 43, wherein training of the machine learning model comprises
displaying dynamic visual neuromodulatory codes to subjects with different types of retinal degeneration disorders; and
measuring neural responses from the subjects during said displaying.

45. A non-transitory computer-readable medium storing instructions that, when executed by one or more processors of a device, cause said one or more processors to perform a method for treating eye disorders, the method comprising
retrieving one or more adapted visual neuromodulatory codes, said one or more adapted visual neuromodulatory codes being adapted to produce neuromodulatory responses in the visual cortex; and
outputting to an electronic display of the device viewable by a patient said one or more adapted visual neuromodulatory codes.

46. The computer-readable medium of claim 45, wherein said one or more adapted visual neuromodulatory codes are adapted to improve processing of visual information in an intact peripheral visual field.

47. The computer-readable medium of claim 46, further comprising performing drug therapy to slow progression of a disorder and/or improve visual function, wherein the drug therapy is designed to reduce growth of abnormal blood vessels and leakage of fluid in the macula.

48. The computer-readable medium of claim 47, wherein the drug therapy comprises anti-vascular endothelial growth factor (VEGF) agents.

49. The computer-readable medium of claim 45, wherein said one or more adapted visual neuromodulatory codes are generated by:
analyzing a first set of brain activity data to produce at least one parameter indicative of an effectiveness of a candidate dynamic visual neuromodulatory code with respect to a participant;
performing, based at least in part on the at least one parameter indicative of an effectiveness of the candidate dynamic visual neuromodulatory code, one of:
(i) outputting the candidate dynamic visual neuromodulatory code as an adapted dynamic visual neuromodulatory code; and
(ii) perturbing the candidate dynamic visual neuromodulatory code, displaying to the participant the perturbed candidate dynamic visual neuromodulatory code, receiving a second set of brain activity data measured during the displaying to the participant the perturbed candidate dynamic visual neuromodulatory code, and iteratively repeating said analyzing using the second set of brain activity data to produce at least one parameter indicative of an effectiveness of the candidate dynamic visual neuromodulatory code with respect to the participant.

50. The computer-readable medium of claim 49, wherein said perturbing comprises obtaining a further candidate dynamic visual neuromodulatory code from a machine learning model trained to target specific neural circuits of the visual cortex to treat retinal degeneration disorders.

51. The computer-readable medium of claim 50, wherein training of the machine learning model comprises
displaying dynamic visual neuromodulatory codes to subjects with different types of retinal degeneration disorders; and
measuring neural responses from the subjects during said displaying.
